(19) United States
(12) Patent Application Publication
(10) Pub. No.: US 2024/0390152 A1
(43) Pub. Date: Nov. 28, 2024

(54) JOINT ENDOPROSTHESIS - IN PARTICULAR, KNEE ENDOPROSTHESIS - AND SYSTEM COMPRISING A JOINT ENDOPROSTHESIS

(71) Applicant: Aesculap AG, Tuttlingen (DE)

(72) Inventors: Arthur Bollinger, Tuttlingen (DE); Frederick Lenzenhuber, Tuttlingen (DE); Berna Richter, Muehlheim (DE)

(21) Appl. No.: 18/639,126

(22) Filed: Apr. 18, 2024

(30) Foreign Application Priority Data
Apr. 20, 2023 (DE) 10 2023 110 011.4

(51) Int. Cl. A61F 2/38; A61F 2/46; A61F 2/48

(52) U.S. Cl. CPC A61F 2/3868; A61F 2/389; A61F 2/482; A61F 2/488; A61F 2002/4666

(57) ABSTRACT

A joint endoprosthesis is made at least partially of or includes a plastic, such as polyether ether ketone (PEEK) or polyether ether ketone (PEEK) composite material. The joint endoprosthesis includes at least one sensor device for sensing a force acting upon a sliding surface of the joint endoprosthesis. The joint endoprosthesis also includes electronics for obtaining the data sensed by the at least one sensor device, and an energy supply device for supplying the at least one sensor device and the electronics. The at least one sensor device is embedded in the joint endoprosthesis in plastic.

### CROSS-REFERENCE TO RELATED APPLICATION(S)

This application claims priority under 35 U.S.C. § 119 to German Application No. 10 2023 110 011.4, filed on Apr. 20, 2023, the content of which is incorporated by reference here in its entirety.

### FIELD

The disclosure relates to a joint endoprosthesis—in particular, a knee endoprosthesis.

### BACKGROUND

Joint endoprostheses—in particular, knee endoprostheses—are known from the prior art. From the prior art, instruments or implant components which are implanted in the patient's joint area temporarily, e.g., during the surgery or before implantation of the actual prosthesis, are also known. For example, WO2021/014313 A1 discloses a spacer component intended to be temporarily implanted in the joint area for the replacement of a joint prosthesis and for the preservation of the dimensions or spaces of the patient's joint area before the implantation of a new prosthesis. The spacer component comprises, for example, various sensors that enable the sensing of data, e.g., a temperature, during the temporary time period.

### SUMMARY

The object of the present disclosure is to provide a joint endoprosthesis that allows the determination of a joint load in vivo.

This object is achieved by a joint endoprosthesis made at least partially of a plastic—in particular, polyether ether ketone (PEEK) or polyether ether ketone (PEEK) composite material- or comprises plastic—in particular, polyether ether ketone (PEEK) or polyether ether ketone (PEEK) composite material.

The selected plastic is preferably a biocompatible plastic. A plastic having a modulus of elasticity very similar or almost identical to the modulus of elasticity of human bone is, advantageously, selected. For example, PEEK has such a similar modulus of elasticity. In this way, the forces acting in the joint under load can be transmitted to a sensor device without distortion. This is not readily possible in the case of metal implants, for example. The modulus of elasticity of metal differs greatly from the modulus of elasticity of human bone.

Furthermore, the joint endoprosthesis comprises at least one sensor device—in particular, pressure sensor—for sensing a force acting upon a sliding surface of the joint endoprosthesis—in particular, a compressive force-electronic means for obtaining the data sensed by the at least one sensor device, and an energy supply device for supplying the at least one sensor device and the electronic means. The sensor device—in particular, pressure sensor—for sensing the force that acts—in particular, under load—upon the sliding surface of the joint endoprosthesis is, for example, a spring-body force sensor, wherein a deformation of the spring body is sensed by means of strain gauges, or a piezoelectric force sensor, wherein a voltage can be sensed when a piezoelectric material is deformed.

The electronic means comprise, for example, a memory device for storing—in particular, temporarily storing—the data sensed by the sensor device, and/or a data transmission device for transmitting the stored or the sensed data. The electronic means also comprise, for example, electrical connections between the sensor device and/or the memory device and/or the data transmission device for transmitting the sensed data between the sensor device and/or the memory device and/or the data transmission device. The energy supply device is, for example, an energy store—in particular, a chargeable energy store—e.g., a battery or an accumulator, and optionally also electrical connections for connecting the energy store to the sensor device.

According to the disclosure, it is also provided that the at least one sensor device be embedded in the joint endoprosthesis—in particular, in plastic—in particular, polyether ether ketone (PEEK) or polyether ether ketone (PEEK) composite material. For example, a corresponding cavity in which the sensor device is inserted is provided in the joint endoprosthesis. For example, the cavity is provided during the production of the joint endoprosthesis—for example, by means of injection molding and/or 3-D printing. Alternatively, the sensor device can already be integrated—molded into the plastic and/or overmolded with the plastic, so to speak—into the joint endoprosthesis during the injection molding or the 3-D printing of the joint endoprosthesis.

In this way, the sensor device can be provided in the region of the joint endoprosthesis sliding surface at which the acting force is to be sensed. By means of the plastic material of the joint endoprosthesis having the modulus of elasticity very similar or almost identical to the modulus of elasticity of human bone, the force acting in the joint at the sliding surface under load can be transmitted to the sensor device without distortion and directly, and can be sensed by the sensor device.

It can also be advantageous if the energy supply device or parts of the energy supply device and/or the electronic means or parts of the electronic means are embedded in the joint endoprosthesis—in particular, in plastic—in particular, polyether ether ketone (PEEK) or polyether ether ketone (PEEK) composite material. For example, corresponding cavities are provided for this purpose. For example, the corresponding parts can already be integrated-molded, so to speak-into the joint endoprosthesis during the injection molding of the joint endoprosthesis.

According to an advantageous embodiment, the energy supply device comprises an energy store and means for charging the energy store—in particular, means for inductive charging. For example, the means for inductive charging comprise a receiving coil, in which a voltage can be induced by means of a transmitting coil, which can be brought into proximity outside the patient's body. In this way, the energy store can be charged—in particular, as needed.

According to an advantageous embodiment, the joint endoprosthesis—in particular, the electronic means of the joint endoprosthesis—comprises a data transmission device, wherein the data transmission device is designed to transmit data wirelessly—in particular, to an external receiving device. The wireless data transmission takes place, for example, by means of Bluetooth-based or NFC-based data transmission. Data are transmitted, for example, to a mobile radio device.

The data can be used for research and science purposes and for further development. The data about the acting forces and, optionally, additional data can be used for further development and for research into causes—for example, in the event of malfunction or fracture of endoprostheses.

According to a preferred embodiment, the joint endoprosthesis is a knee joint endoprosthesis or forms at least a part of a knee joint endoprosthesis. The joint endoprosthesis is or comprises at least one first joint component—for example, a femoral component or a tibial component or a meniscal component. The joint endoprosthesis can also comprise a femoral component, a tibial component, and a meniscal component—in particular, a meniscal component molded onto the tibial component. Advantageously, at least the femoral component is made of plastic—in particular, PEEK or a PEEK composite material.

The first joint component of the joint endoprosthesis is, for example, a femoral component, wherein the femoral component comprises at least one femoral condyle. In a distal region of the femoral condyle, at least one sensor device for sensing a force acting upon a sliding surface of a joint endoprosthesis is provided. The femoral condyle comprises a femoral joint surface, wherein the femoral joint surface forms a sliding surface of the joint endoprosthesis, and the force acting upon the femoral joint surface in the distal region of the femoral condyle can be sensed by means of the sensor device. In the case of a unilateral surface replacement, the femoral component comprises either a lateral or a medial femoral condyle. In the case of a total surface replacement, the femoral component comprises either a lateral or a medial femoral condyle. In this case, at least one sensor device is provided in a femoral condyle in question in a corresponding distal region of the femoral condyle. In the knee joint, the tibio-femoral force transmission takes place via the medial femoral condyle and the lateral femoral condyle. By providing the sensors on both sides, the acting forces can be sensed separately at respective condyles.

The femoral component comprises, for example, a retropatellar slide groove, trochlea. It can be advantageous if at least one part of the energy supply device, e.g., a receiving coil for inductive charging, is provided in the region of the trochlea. In this way, a transmitting coil, which can be brought into proximity outside the patient's body, can be brought comparatively close to the receiving coil.

The first joint component of the joint endoprosthesis is, for example, a femoral component, wherein the femoral component comprises at least one femoral condyle and/or trochlea. Advantageously, a sensor device or an additional sensor device for sensing a force acting upon a sliding surface of the joint endoprosthesis can be provided in a posterior region of the femoral condyle, and/or a sensor device or an additional sensor device for sensing a force acting upon a sliding surface of the joint endoprosthesis can be provided in the region of the trochlea. In the posterior region of the condyles, relatively large forces act upon the femoral joint surfaces in a patient during bending of the knee joints—for example, during stair climbing. In the region of the trochlea, a force that acts at the surface between the trochlea and the patella—also called patellar pressure—can be measured. Values of the patellar pressure can indicate correct or incorrect positioning of the endoprosthesis. Basically, to this day, there has been little research into the patellar pressure or the pressure profile. The sensed data can provide important findings for research and science. For example, the data can be used to improve the joint endoprosthesis.

It can be provided that the joint endoprosthesis comprise at least one sensor device for detecting an infection in the joint endoprosthesis and/or in an implantation region of the joint endoprosthesis—in particular, by sensing C-reactive protein—and/or at least one sensor device for sensing a temperature, and/or at least one sensor device for sensing an acceleration. By means of a CRP sensor, it is possible, for example, to sense the presence or a concentration of C-reactive protein. C-reactive protein can indicate an infection. By means of a temperature sensor, a temperature is sensed, for example. An increase in the temperature or an elevated temperature can likewise indicate an infection.

The acceleration sensor senses, for example, a motion or an acceleration. For example, the data sensed by the acceleration sensor are used to operate electronics of the joint endoprosthesis in an inactive mode and in an active mode, wherein the electronics are able to be switched between the inactive mode and the active mode according to data sensed by means of an acceleration sensor. For example, the active mode can be activated on the basis of the sensing of a motion/acceleration. In the active mode, data are sensed, for example, by means of the pressure sensor and, optionally, by means of additional sensors present, e.g., a CRP sensor, a temperature sensor, and, optionally, additional pressure sensors, and, optionally, transmitted by means of the data transmission device. For example, the acceleration sensor can also detect very small, barely visible movements, also known as micro-movements. Such micro-movements indicate, for example, unintentional loosening of the joint endoprosthesis.

If no motion/acceleration is detected, e.g., over a defined period of time, the inactive mode can be activated, for example. In the inactive mode, no data are sensed by means of the pressure sensor and, optionally, by means of additional sensors present, e.g., a CRP sensor, a temperature sensor, and, optionally, additional pressure sensors, and, optionally, also no data are transmitted.

It can be provided that the joint endoprosthesis comprise a shaft, which can be inserted into a bone cavity, wherein the energy supply device or parts of the energy supply device and/or the electronic means or parts of the electronic means are arranged in the shaft.

Further embodiments relate to a method for sensing a force acting upon a sliding surface of a joint endoprosthesis—in particular, a compressive force—by means of a joint endoprosthesis according to the described embodiments. The method provides that a force acting upon a sliding surface of the joint endoprosthesis—in particular, a compressive force—be sensed by means of a sensor device—in particular, a pressure sensor—wherein the sensed data are obtained from the at least one sensor device by means of electronic means, wherein the sensor device and the electronic means are supplied by means of an energy supply device, and wherein the sensor device is embedded in the joint endoprosthesis—in particular, in plastic—in particular, polyether ether ketone (PEEK) or polyether ether ketone (PEEK) composite material.

The data can be provided for research and science purposes and for further development. The data about the acting forces and, optionally, additional data can be used for further development and for research into causes—for example, in the event of malfunction or fracture of endoprostheses.

According to one embodiment of the method, the sensed data are transmitted by means of a data transmission device, and items of information that can be displayed by means of a display device are provided on the basis of the transmitted data. The items of information that can be displayed can also provide information to a patient, for example.

According to one embodiment of the method, electronics of the joint endoprosthesis can be operated in an inactive mode and in an active mode, and switching between the inactive mode and the active mode is carried out according to data sensed by means of an acceleration sensor. The active mode can be activated on the basis of the sensing of a motion/acceleration. In the active mode, data are sensed, for example, by means of the pressure sensor and, optionally, by means of additional sensors present, e.g., a CRP sensor, a temperature sensor, and, optionally, additional pressure sensors, and, optionally, transmitted by means of the data transmission device.

If no motion/acceleration is detected, e.g., over a defined period of time, the inactive mode is activated. In the inactive mode, no data are sensed by means of the pressure sensor and, optionally, by means of additional sensors present, e.g., a CRP sensor, a temperature sensor, and, optionally, additional pressure sensors, and, optionally, also no data are transmitted.

Further embodiments relate to a system comprising a joint endoprosthesis according to the described embodiments and a display device for displaying items of information on the basis of data sensed by means of the joint endoprosthesis. The display device is, for example, a display of a mobile radio device that receives data from the joint endoprosthesis.

Further embodiments relate to a computer program product having a computer program which, by execution in a computing device, causes a display device of a system to display items of information on the basis of data sensed by means of the joint endoprosthesis. The computing device is, for example, a processor of a mobile radio device that receives data from the joint endoprosthesis.

Further embodiments relate to a manufacturing method for manufacturing a joint endoprosthesis, in particular a knee endoprosthesis, for example a femoral component or a tibial component according to the embodiments described above. According to the method, it is provided that the production comprises an injection molding process or a 3-D printing process, wherein at least one sensor device is overmolded with a plastic, for example polyether ether ketone (PEEK) or polyether ether ketone (PEEK) composite material, in an injection molding process or is embedded in the plastic, for example polyether ether ketone (PEEK) or polyether ether ketone (PEEK) composite material, in a 3-D printing process. The sensor device is therefore advantageously embedded directly in the shaping manufacturing step of the joint endoprosthesis. In particular, the sensor device is not subsequently the shaping manufacturing step of the joint endoprosthesis embedded in the joint endoprosthesis or mounted on the joint endoprosthesis.

### BRIEF DESCRIPTION OF THE DRAWINGS

Further advantages are found in the description and the attached drawings. Embodiments of the disclosure are shown in the drawings and explained in more detail in the following description. In this case, the same reference signs in different drawings denote the same or at least functionally comparable elements. In the description of individual drawings, reference may also be made to elements from other drawings. The figures show the following in schematic form:

FIG. 1a shows a first embodiment of a joint endoprosthesis according to the disclosure in a first view;

FIG. 1b shows the first embodiment in a second view;

FIG. 1c shows the first embodiment in a third view;

FIG. 2a shows a part of the joint endoprosthesis according to FIGS. 1a-1c in a first view, said part forming a joint body;

FIG. 2b shows the part according to FIG. 2a in a second view;

FIG. 3 shows electronic components of the joint endoprosthesis;

FIG. 4 shows a portion of the electronic components of FIG. 3 of the joint endoprosthesis in an exploded view;

FIG. 5a shows a second embodiment of a joint endoprosthesis according to the disclosure in a first view;

FIG. 5b shows the second embodiment in a second view;

FIG. 6a shows a third embodiment of a joint endoprosthesis according to the disclosure in a first view;

FIG. 6b shows the third embodiment in a second view;

FIG. 7 shows electronic components of the joint endoprosthesis according to FIGS. 6a and 6b; and

FIG. 8 shows a display device.

### DETAILED DESCRIPTION

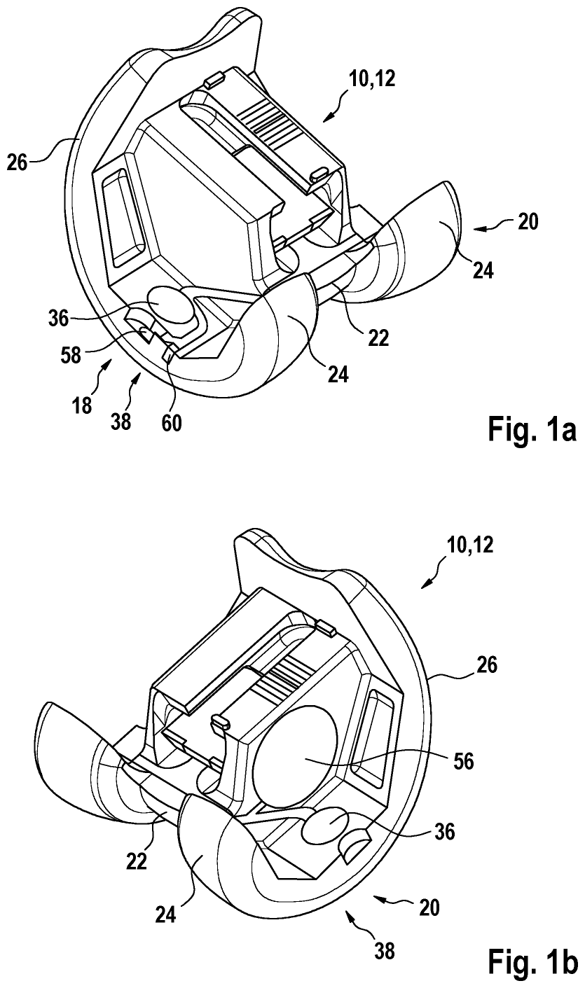

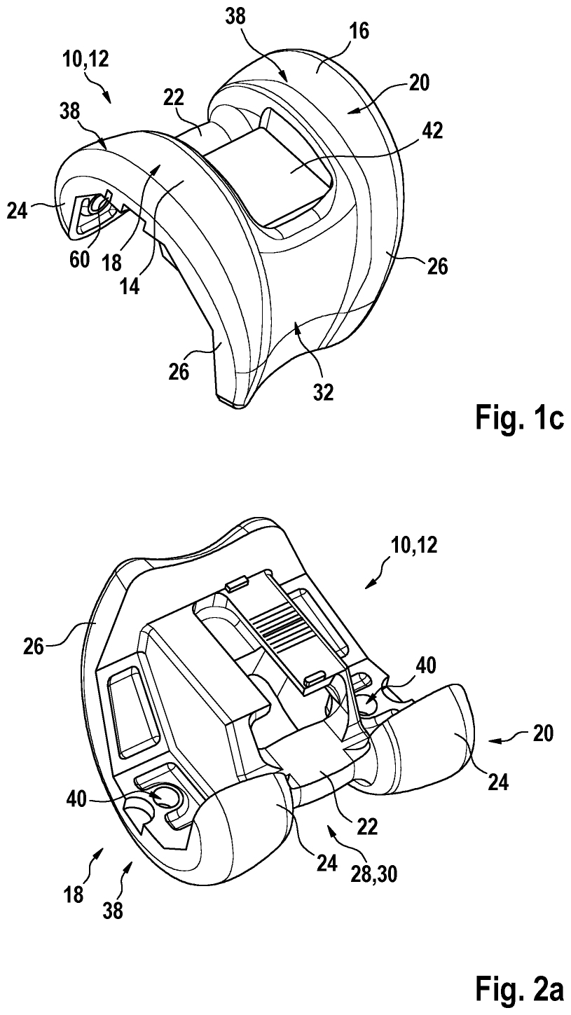

FIGS. 1a-1c show a joint endoprosthesis according to the disclosure. The joint endoprosthesis in its entirety is provided with the reference sign 10. In the example, the joint endoprosthesis 10 is a knee endoprosthesis. In the example, the knee endoprosthesis 10 comprises a femoral component 12, which can be anchored to a femur bone. The knee endoprosthesis 10 can also comprise a tibial component and a meniscal component—in particular, a meniscal component formed on the tibial component (not shown in the figures).

In the example, the femoral component 12 comprises a medial and a lateral femoral joint surface 14 and 16, which form outer surfaces of two femoral condyles 18 and 20, viz., a medial condyle 18 and a lateral condyle 20, of the femoral component 12. The femoral joint surfaces 14 and 16 are designed correspondingly or substantially correspondingly for cooperation with meniscal joint surfaces of the meniscal component.

It can be advantageous if the femoral joint surfaces 14 and 16 each form a portion of a spherical surface. The femoral joint surfaces 14 and 16 are designed, together with the meniscal joint surfaces, in such a way that the joint surfaces seated against one another in each case slide and/or roll on one another.

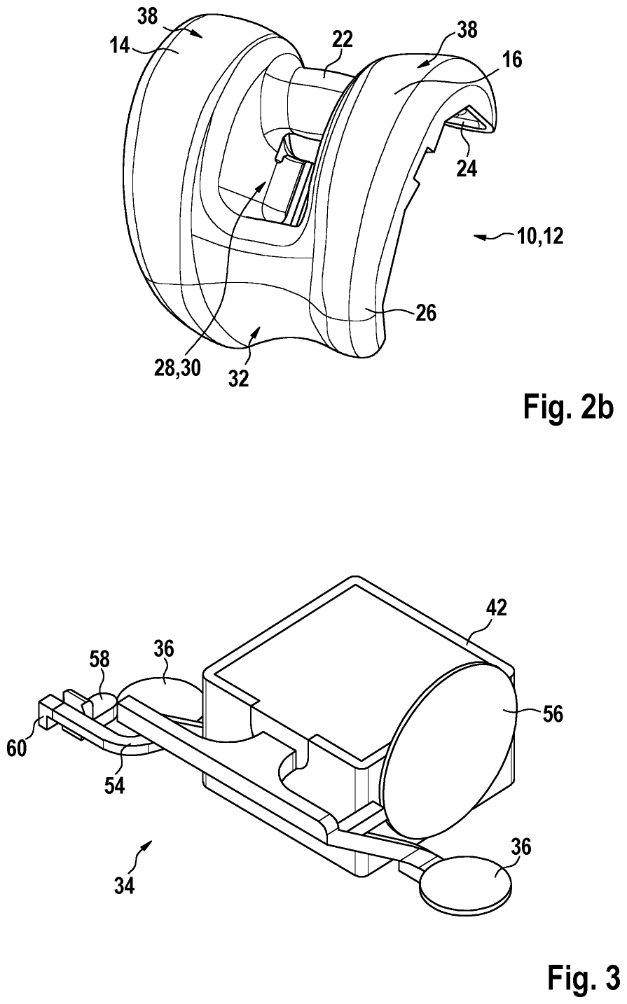

In the example, a stabilizing element 22 is arranged between posterior ends 24 of the condyles 18 and 20 and connects them to one another in the lateral-medial direction. Anterior ends 26 of the condyles 18 and 20 are directly connected to one another. Overall, a void 28 in the form of a through-hole 30 is thereby defined, which is delimited laterally and anteriorly by the condyles 18 and 20 and posteriorly by the stabilizing element 22; cf. FIGS. 2a and 2b.

In the region of the anterior ends 26 of the condyles 18 and 20, a trochlear region 32, also referred to as trochlear slide groove or trochlea, is formed.

In the example, the femoral component 12 in itself, i.e., the part forming the physical shape of the femoral component 12, is made of plastic—for example, polyether ether ketone (PEEK) or polyether ether ketone (PEEK) composite material. The production can take place, for example, in an injection molding process or a 3-D printing process.

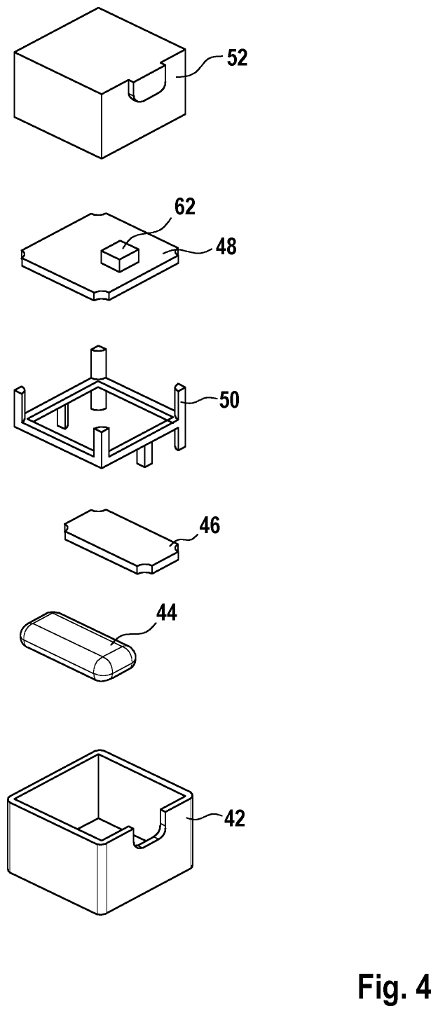

In the example, the joint endoprosthesis 10 comprises electronic components 34. In FIG. 3, the electronic components 34 are shown separately. In FIGS. 1a-1c, the electronic components 34 are arranged on or partially also in the joint endoprosthesis 10.

According to FIGS. 1a, 1b and 3, the electronic components 34 comprise two sensor devices 36 for sensing forces acting upon sliding surfaces of the joint endoprosthesis 10—in particular, compressive forces. The sensor devices 36 are also referred to in simplified form as pressure sensors 36. In the example, one pressure sensor 36 is arranged in a distal region 38 of each condyle 18 and 20. In the distal region 38 of the condyles, relatively large forces act upon the femoral joint surfaces 14 and 16 in a patient during standing and during walking motion.

In the example, the pressure sensors 36 are embedded in corresponding cavities 40 in the joint endoprosthesis 10. The cavities 40 are also shown, for example, in FIG. 2a, which shows the joint endoprosthesis 10 without the electronic components 34.

The electronic components 34 also comprise electronic means for obtaining the data sensed by the pressure sensors 36, and an energy supply device for supplying the pressure sensors and the electronic means.

In the example, a portion of the electronic components is arranged in a housing 42; cf. FIGS. 3 and 4. An energy store 44, two circuit boards 46, 48—in particular, with additional electronics not shown in detail—and support elements 50 are arranged, together with potting compound 52, in the housing. For example, a data memory—in particular, for storing and/or temporarily storing the data sensed by the pressure sensors 36—and a data transmission device for transmitting the stored or the sensed data are also provided in the housing 42. The data transmission device is designed to transmit data wirelessly—in particular, to an external receiving device. The wireless data transmission takes place, for example, by means of Bluetooth-based or NFC-based data transmission. Data are transmitted, for example, to a mobile radio device.

According to the example, the pressure sensors 36 are connected to the electronics in the housing 42—in particular, to the energy store, to the data memory, and/or to the data transmission device—by corresponding electronic connection means 54. For example, by means of the connection means 54, energy is supplied from the energy store 44 and/or data are transmitted between pressure sensors 36 and the data memory and/or the data transmission device.

In the example, besides the pressure sensors 36, others of the electronic components, e.g., a portion of the electronic connection means 54, are embedded in corresponding cavities in the joint endoprosthesis 10. The cavities are provided, for example, during the production of the joint endoprosthesis—for example, by means of injection molding and/or 3-D printing. Alternatively, the corresponding electronic components can already be integrated—molded, so to speak—into the joint endoprosthesis 10 during the injection molding of the joint endoprosthesis 10. The joint endoprosthesis 10 also comprises a cavity for the housing 42, in the region of a shaft (the shaft is not shown).

According to the example, the joint endoprosthesis 10 comprises a receiving coil 56 for charging the energy store 44 by means of inductive charging. A voltage can be induced in the receiving coil by means of a transmitting coil, which can be brought into proximity outside the patient's body. In this way, the energy store 44 can be charged—in particular, as needed.

According to the example, the electronic components 34 of the joint endoprosthesis 10 comprise additional sensing equipment.

For example, sensors that enable early detection of infections are provided. By means of a CRP sensor 58, it is possible, for example, to sense the presence or a concentration of C-reactive protein. By means of a temperature sensor 60, a temperature is sensed, for example. An increase in the temperature or an elevated temperature can likewise indicate an infection.

Additional pressure sensors can also be provided (not shown in the figures). For example, a pressure sensor or a plurality of pressure sensors can be provided in a region of the posterior ends 24, 26 of the condyles 18, 20. In this region, relatively large forces act upon the femoral joint surfaces 12, 16 in a patient during bending of the knee joints—for example, during stair climbing. For example, one or more pressure sensors can be provided in the region of the trochlea 32. In this region, a force that acts at the surface between the trochlea and the patella—also called patellar pressure—can be measured. Values of the patellar pressure can indicate correct or incorrect positioning of the endoprosthesis.

In the example, the joint endoprosthesis 10 comprises an acceleration sensor 62. The acceleration sensor 62 senses, for example, a motion or an acceleration. For example, the data sensed by the acceleration sensor 62 are used to operate electronics of the joint endoprosthesis in an inactive mode and in an active mode, wherein the electronics are able to be switched between the inactive mode and the active mode according to data sensed by means of an acceleration sensor.

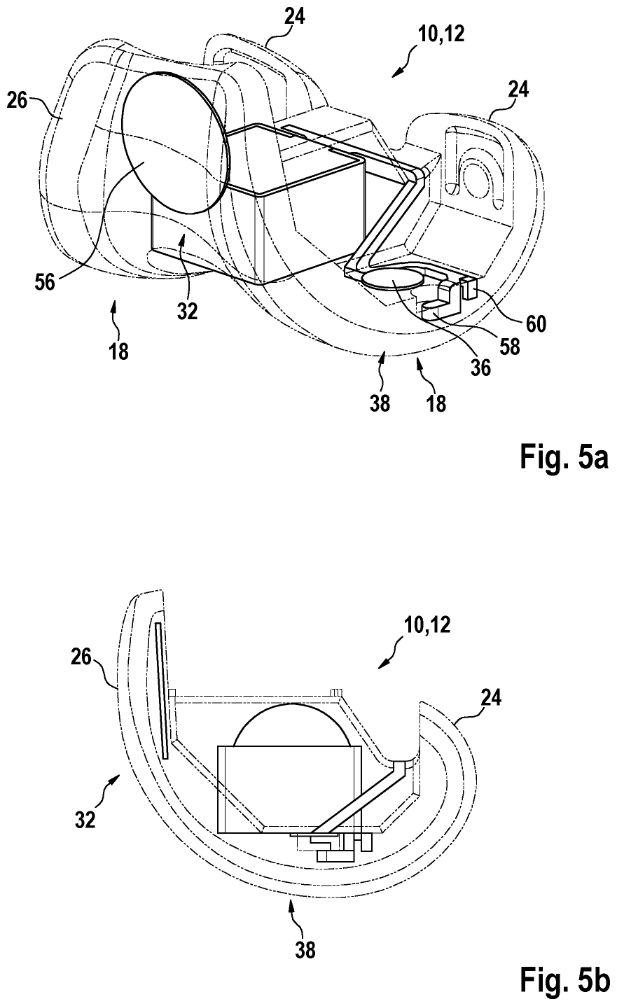

FIGS. 5a and 5b show a joint endoprosthesis 10 according to another embodiment. The receiving coil 56 is arranged in the region of the trochlea 32.

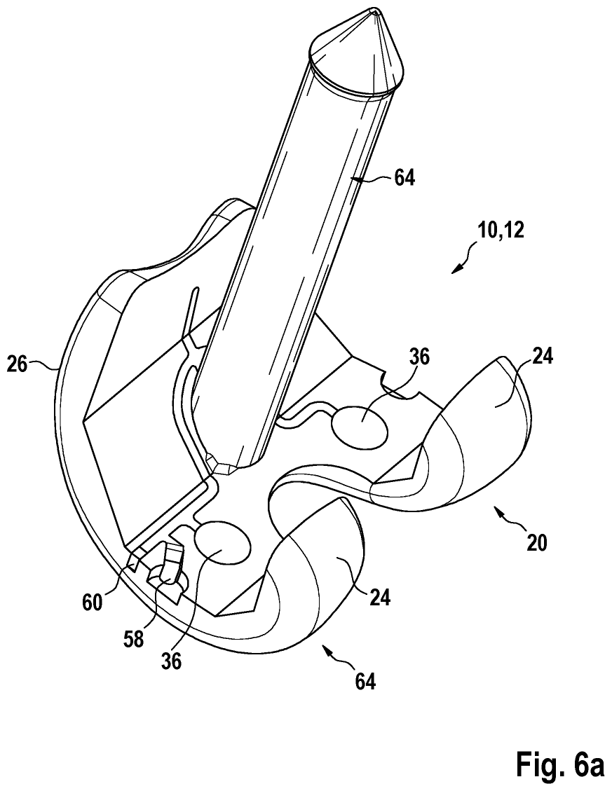

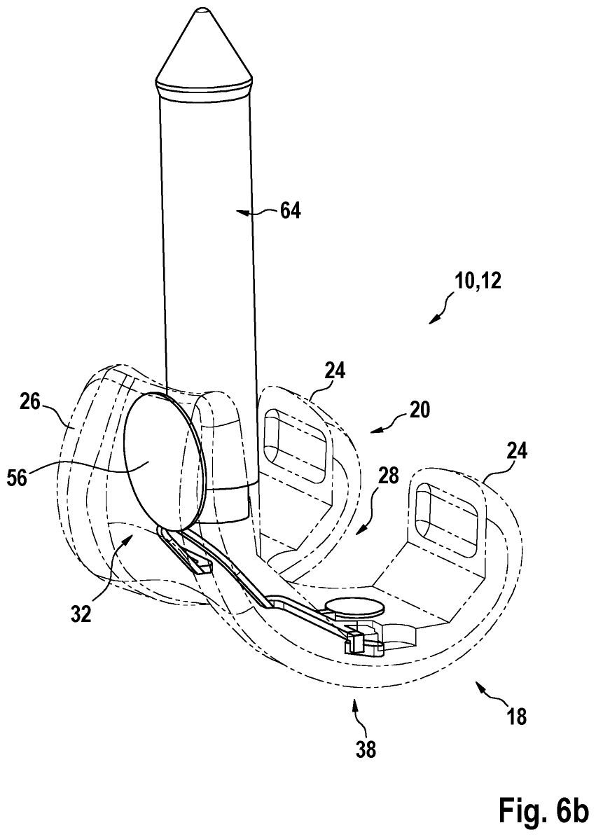

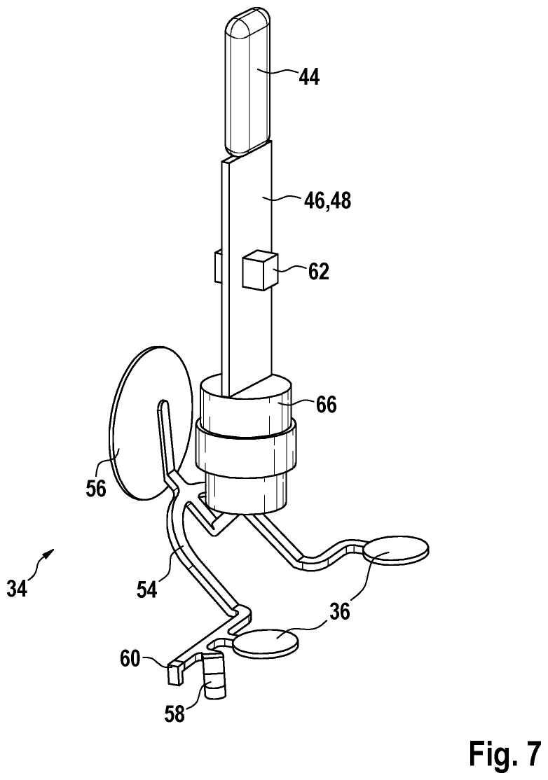

FIGS. 6a and 6b show a joint endoprosthesis 10 according to another embodiment. The joint endoprosthesis 10 comprises a shaft 64, which can be inserted into a bone cavity—in the example, into a femur bone. In the example, a portion of the electronic components 34 is arranged in the shaft 64. FIG. 7 shows the electronic components without the shaft 64. By means of an adapter part 66, the portion of the electronic components arranged in the shaft can be coupled to the portion of the electronic components arranged outside the shaft. The shaft 64 forms a housing for the electronics arranged therein; cf. FIGS. 6a and 6b.

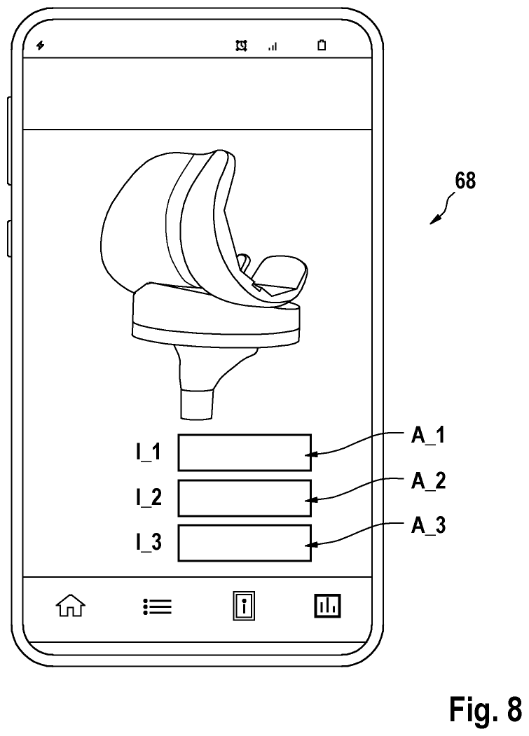

FIG. 8 shows a display device 68. The display device 68 is, for example, a mobile radio device. For example, the display device 68 receives data from the data transmission device of the joint endoprosthesis 10. By means of a suitable application, or app, items of information are displayed on a display of the display device 68 on the basis of the received data. For example, three different items of information I_1, I_2, and I_3 are shown in a total of three display fields. In a first display field A_1, an item of information about a temperature sensed by means of the temperature sensor 60 of the joint endoprosthesis 10 is shown, for example. In a second display field A_2, an item of information about force or compressive force sensed by means of the pressure sensor 36 of the joint endoprosthesis 10 is shown, for example. In a third display field A 3, an item of information about a CRP concentration sensed by means of the CRP sensor 58 of the joint endoprosthesis 10 is shown, for example. The items of information can be displayed as absolute values or, according to corresponding limit values, as OK, critical, or not OK—optionally in combination with indications and/or colors—for example, green, orange, red. The application can assist, for example, in rehabilitation or recovery. For example, the patient can be informed about the present load on the joint by the display of the items of information. The use can also be combined with items of information about permissible loads and flexion angles. By using these items of information, the patient can learn and improve the handling of the joint endoprosthesis 10.

1. A joint endoprosthesis made at least partially of a plastic or comprises plastic, the joint endoprosthesis comprising:
at least one sensor device for sensing a force acting upon a sliding surface of the joint endoprosthesis;
electronics for obtaining data sensed by the at least one sensor device; and
an energy supply device for supplying power to the at least one sensor device and the electronics, and wherein the at least one sensor device is embedded in the joint endoprosthesis.

2. The joint endoprosthesis according to claim 1, wherein the energy supply device or parts of the energy supply device and/or the electronics or parts of the electronics are embedded in the joint endoprosthesis.

3. The joint endoprosthesis according to claim 1, wherein the energy supply device comprises an energy store and a charger for charging the energy store.

4. The joint endoprosthesis according to claim 1, wherein the joint endoprosthesis comprises a data transmission device, wherein the data transmission device is designed to transmit data wirelessly.

5. The joint endoprosthesis according to claim 1, wherein the joint endoprosthesis is a knee joint endoprosthesis or forms at least a part of a knee joint endoprosthesis, wherein the joint endoprosthesis is or comprises at least one first joint component.

6. The joint endoprosthesis according to claim 5, wherein the at least one first joint component is a femoral component, and the femoral component comprises at least one femoral condyle, wherein at least one sensor device is provided in a distal region of the femoral condyle, wherein the femoral condyle comprises a femoral joint surface, wherein the femoral joint surface forms the sliding surface of the joint endoprosthesis, and wherein the force acts upon the sliding surface in a distal region of the femoral condyle, the force being detectable by the sensor device.

7. The joint endoprosthesis according to claim 5, wherein the first joint component is a femoral component, and the femoral component comprises at least one retropatellar slide groove, trochlea, and wherein at least one part of the energy supply device is provided in a region of the trochlea.

8. The joint endoprosthesis according to claim 5, wherein the first joint component is a femoral component, and the femoral component comprises at least one femoral condyle and/or trochlea, and wherein a sensor device or an additional sensor device for sensing the force acting upon the sliding surface of the joint endoprosthesis is provided in a posterior region of the femoral condyle, and/or wherein a sensor device or an additional sensor device for sensing the force acting upon the sliding surface of the joint endoprosthesis is provided in a region of the trochlea.

9. The joint endoprosthesis according to claim 1, wherein the joint endoprosthesis comprises at least one infection sensor device for detecting an infection in the joint endoprosthesis and/or in an implantation region of the joint endoprosthesis and/or at least one temperature sensor device for sensing a temperature, and/or at least one acceleration sensor device for sensing an acceleration.

10. The joint endoprosthesis according to claim 1, wherein the joint endoprosthesis comprises a shaft that is insertable into a bone cavity, wherein the energy supply device or parts of the energy supply device and/or the electronics or parts of the electronics are arranged in the shaft.

11. A method for sensing a force acting upon a sliding surface of the joint endoprosthesis according to claim 1, the method comprising the steps of:
sensing a force acting upon the sliding surface of the joint endoprosthesis via the at least one sensor device; and
obtaining sensed data from the at least one sensor device via the electronics.

12. The method according to claim 11, wherein the sensed data are transmitted via a data transmission device, and wherein items of information that are displayable on a display device are provided based on the sensed data.

13. The method according to claim 11, wherein the electronics are operable in an inactive mode and in an active mode, and wherein switching between the inactive mode and the active mode is carried out according to data sensed via an acceleration sensor.

14. A system comprising:
the joint endoprosthesis according to claim 1; and
a display device for displaying items of information based on data sensed by the joint endoprosthesis.

15. A computer program product having a computer program which, by execution in a computing device, causes the display device of the system according to claim 14 to display the items of information based on the data sensed by the joint endoprosthesis.
